(19) United States
(12) Patent Application Publication
(10) Pub. No.: US 2025/0273449 A1
(43) Pub. Date: Aug. 28, 2025

(54) Data Processing Method, Information Processing Device and Computer Readable Medium

(71) Applicant: SHIMADZU CORPORATION, Kyoto-shi (JP)

(72) Inventor: Kaori SUGIMURA, Kyoto-shi (JP)

(21) Appl. No.: 19/002,821

(22) Filed: Dec. 27, 2024

(30) Foreign Application Priority Data
Feb. 26, 2024 (JP) 2024-026185

(51) Int. Cl. H01J 49/00; G06F 17/18

(52) U.S. Cl. CPC H01J 49/0036; G06F 17/18

(57) ABSTRACT

A data processing method is performed by a computer to correct measurement values of a prescribed component obtained by measurement with an analysis device. The data processing method includes: receiving the measurement values; and calculating a first reference value and a second reference value using the measurement values. The data processing method includes correcting the measurement values using the first reference value and the second reference value.

### CROSS REFERENCE TO RELATED APPLICATIONS

This nonprovisional application is based on Japanese Patent Application No. 2024-026185 filed on Feb. 26, 2024 with the Japan Patent Office, the entire contents of which are hereby incorporated by reference.

### BACKGROUND OF THE INVENTION

### Field of the Invention

The present disclosure relates to a data processing method, an information processing device and a computer readable medium, and more particularly to data processing for correcting measurement values obtained by an analysis device.

### Description of the Background Art

Measurement values obtained by an analysis device may be affected by a temperature, a humidity, a state of the analysis device, and a device difference between analysis devices during measurement. For example, in a mass spectrometer, contamination of a mass spectrometry portion may affect the measurement sensitivity. Therefore, even when the same sample is measured with the same mass spectrometer, different measurement values may be obtained before and after cleaning of the mass spectrometry portion. Therefore, the measurement values obtained by the analysis device may include a measurement error. Examples of the measurement error include an error caused by a difference in timing of measurement of a sample, an error caused by a difference in environment where a sample is measured, and an error caused by a difference in equipment used in analysis of a sample.

As a method of correcting the measurement error, “Procedures for large-scale metabolic profiling of serum and plasma using gas chromatography and liquid chromatography coupled to mass spectrometry” by Dunn, Warwick B., et al., Nature protocols 6.7 (2011): 1060-1083 discloses the technique for correcting measurement values of samples based on measurement values of a pooled QC prepared by mixing all of the samples in equal amounts. In “Procedures for large-scale metabolic profiling of serum and plasma using gas chromatography and liquid chromatography coupled to mass spectrometry” by Dunn, Warwick B., et al., Nature protocols 6.7 (2011): 1060-1083, the pooled QC is measured in between measurements of the samples. After the measurements, LOESS smoothing is performed using only the measurement values of the pooled QC, to calculate an approximate curve. Based on the calculated approximate curve, the measurement values of the samples are corrected. This method is called “QC-based robust LOESS signal correction (QC-RLSC) method”.

The pooled QC is prepared by mixing all of the samples in equal amounts. Since all of the samples are mixed in equal amounts, an amount of each component included in the pooled QC is an average of all of the samples about this component. That is, the measurement values of the pooled QC are close to an average value of the measurement values of all of the samples, which makes it possible to prevent the measurement values of the pooled QC from significantly deviating from the measurement values of the samples. In addition, since the pooled QC is prepared by mixing all of the samples, a component included in at least any one of the samples is included in the pooled QC. Therefore, even when a component to be subjected to correction is unknown at the start of measurement, measurement values of the component can be subjected to correction processing after measurement.

### SUMMARY OF THE INVENTION

In the method disclosed in “Procedures for large-scale metabolic profiling of serum and plasma using gas chromatography and liquid chromatography coupled to mass spectrometry” by Dunn, Warwick B., et al., Nature protocols 6.7 (2011): 1060-1083, the pooled QC is prepared by mixing all of the samples in equal amounts. Therefore, when the samples are solids, for example, preparation of the pooled QC may be difficult. Thus, there is a demand for a data processing method that does not require preparation of a pooled QC and allows measurement values of samples to be corrected using an equivalent correction method.

The present disclosure has been made in view of the above-described circumstances and an object of the present disclosure is to provide the technique for correcting measurement values obtained by an analysis device, without using a pooled QC.

A data processing method according to a first aspect of the present disclosure is performed by a computer to correct an N-th (N is an integer satisfying 3≤N) measurement value, of a plurality of measurement values of a prescribed component obtained by measuring a plurality of samples with an analysis device. The computer includes a processor and an interface. The data processing method includes: 1) receiving, by the interface, the plurality of measurement values obtained by the analysis device; 2) estimating, by the processor, reference values, the reference values being values obtained when a standard sample obtained by mixing the plurality of samples is measured at the time of two or more measurements including at least M-th and Q-th (M and Q are natural numbers satisfying M<N<Q) measurements; and 3) correcting, by the processor, the N-th measurement value using the reference values. The estimating reference values includes: a) estimating, by the processor, a Q-th reference value using T-th (T is a natural number satisfying T<Q) to Q-th measurement values, the Q-th reference value being a value obtained when the standard sample is measured at the time of the Q-th measurement; and b) estimating, by the processor, an M-th reference value using L-th (L is a natural number satisfying L<M) to M-th measurement values, the M-th reference value being a value obtained when the standard sample is measured at the time of the M-th measurement.

An information processing device according to a second aspect of the present disclosure is an information processing device that corrects a measurement value obtained by measurement with an analysis device. The information processing device includes: at least one or more processors; and a memory configured to access the one or more processors. The memory is configured to store one or more instructions executed by the one or more processors. The processor is configured to, by executing the one or more instructions, receive measurement values of a prescribed component obtained by measuring a plurality of samples with the analysis device, estimate reference values, the reference values being values obtained when a standard sample obtained by mixing the plurality of samples is measured at the time of two or more measurements including at least M-th and Q-th (M and Q are natural numbers satisfying M<Q) measurements, and correct an N-th (N is a natural number satisfying M<N<Q) measurement value using the reference values. The processor is configured to, when estimating the reference values, estimate a Q-th reference value using T-th (T is a natural number satisfying T<Q) to Q-th measurement values, the Q-th reference value being a value obtained when the standard sample is measured at the time of the Q-th measurement, and estimate an M-th reference value using L-th (L is a natural number satisfying L<M) to M-th measurement values, the M-th reference value being a value obtained when the standard sample is measured at the time of the M-th measurement.

A computer readable medium according to a third aspect of the present disclosure is a non-transitory computer readable medium having a program recorded thereon. The program causes a computer to: receive measurement values of a prescribed component obtained by measuring a plurality of samples with an analysis device; estimate reference values, the reference values being values obtained when a standard sample obtained by mixing the plurality of samples is measured at the time of two or more measurements including at least M-th and Q-th (M and Q are natural numbers satisfying M<Q) measurements; and correct an N-th (N is a natural number satisfying M<N<Q) measurement value using the reference values. The program causes the computer to, when estimating the reference values, estimate a Q-th reference value using T-th (T is a natural number satisfying T<Q) to Q-th measurement values, the Q-th reference value being a value obtained when the standard sample is measured at the time of the Q-th measurement, and estimate an M-th reference value using L-th (L is a natural number satisfying L<M) to M-th measurement values, the M-th reference value being a value obtained when the standard sample is measured at the time of the M-th measurement.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 is a schematic diagram of an analysis system according to an embodiment.

FIG. 2 is a diagram for illustrating a procedure of preparing a pooled QC.

FIG. 3 is a diagram for illustrating an order of measurement of the pooled QC and samples.

FIG. 4 is a diagram for illustrating a method of correcting measurement values of samples based on measurement values of a pooled QC in a comparative example.

FIG. 5 is a diagram for illustrating a data processing method according to the embodiment.

FIG. 6 is a diagram for illustrating a data processing method when analysis data is added.

FIG. 7 is a flowchart showing data processing according to the embodiment.

FIG. 8 is a flowchart showing a subroutine of step S20 shown in FIG. 7.

### DESCRIPTION OF THE PREFERRED EMBODIMENTS

Hereinafter, an embodiment of the present disclosure will be described in detail with reference to the drawings. Although a mass spectrometer is described below as an example of an analysis device, the present disclosure is not limited thereto and is applicable to any analysis device. In the drawings, the same or corresponding portions are denoted by the same reference characters and description thereof will not be repeated.

### Overall Configuration of Analysis System

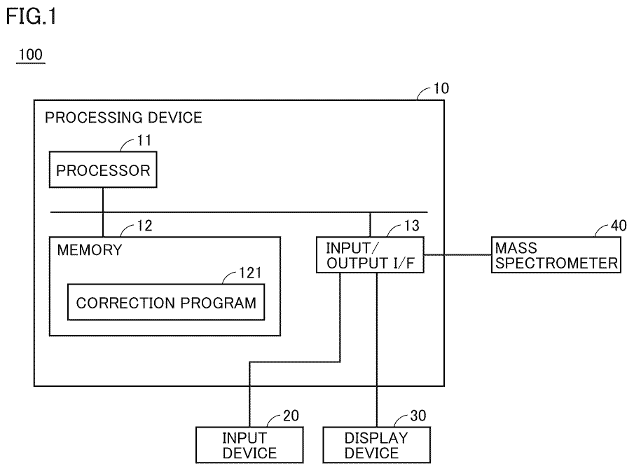

FIG. 1 is a block diagram showing a configuration of an analysis system 100 according to an embodiment. Referring to FIG. 1, analysis system 100 includes a processing device 10, an input device 20, a display device 30, and a mass spectrometer 40. Analysis system 100 corrects a measurement error about measurement values of samples obtained by mass spectrometer 40. Examples of the measurement error include an error caused by a difference in timing of measurement of a sample, an error caused by a difference in environment where a sample is measured, and an error caused by a difference in equipment used in analysis of a sample. Processing device 10 may be incorporated into mass spectrometer 40.

Processing device 10 includes a processor 11, a memory 12 and an input/output interface (I/F) 13. These components are communicatively connected to each other through a bus.

Processor 11 is an example of an electrical circuit and controls the operation of processing device 10 by executing a given program. The program executed by processor 11 may be stored in memory 12, or may be stored in a storage device (not shown) that is external to processing device 10. Processor 11 is, for example, a central processing unit (CPU).

Memory 12 can non-transitorily store the program executed by processor 11, mass spectrum data created by mass spectrometer 40, and measurement values of components. The measurement values of the components are calculated from an area value of a peak in a mass spectrum and a height of the peak in the mass spectrum, for example. The program stored in memory 12 includes a correction program 121. Memory 12 includes a volatile memory (e.g., a random access memory (RAM)) and a non-volatile memory (e.g., a read only memory (ROM), a hard disc drive and a solid state drive). The above-described program may be stored in an external storage device that can be accessed by processor 11.

Input/output I/F 13 is an interface for exchanging various types of data between processor 11 and the devices connected to input/output I/F 13. Input device 20, display device 30 and mass spectrometer 40 are connected to input/output I/F 13. Input/output I/F 13 is implemented by, for example, a terminal block, a connector and a network adapter. Exchanging the data through input/output I/F 13 may be performed wirelessly using Bluetooth (registered trademark), wireless LAN or the like, or may be performed in a wired manner using a universal serial bus (USB) or the like. Processing device 10 can receive, through input/output I/F 13, measurement values obtained by a device other than mass spectrometer 40, and perform data processing on the measurement values.

Input device 20 receives input of information from a user to processing device 10. The information includes, for example, the total number of samples, types of samples, and a component to be measured. Input device 20 is implemented by, for example, a touch panel, a mouse and a keyboard.

Display device 30 displays information in accordance with an instruction from processing device 10. The information includes, for example, a mass spectrum of a sample, measurement values of a prescribed component before correction, and measurement values of the prescribed component after correction. Display device 30 is implemented by, for example, a liquid crystal display that can display an image.

Mass spectrometer 40 analyzes a sample and creates mass spectrum data indicating mass distributions of ions derived from components included in the sample. The created mass spectrum data is transmitted to processing device 10. Processing device 10 calculates measurement values of each component from the mass spectrum data. Mass spectrometer 40 is, for example, a liquid chromatography mass spectrometer, a gas chromatography mass spectrometer, a high performance liquid chromatography-tandem mass spectrometer, and a gas chromatography-tandem mass spectrometer.

### Comparative Example

In measurement using a mass spectrometer, contamination may occur in a mass spectrometry portion in the process of repeated measurement and the contamination may affect an analysis result. For example, even if the same sample is measured, measurement values obtained by measuring the sample on different days do not match with each other in some cases. That is, the measurement values obtained by the mass spectrometer may include a measurement error.

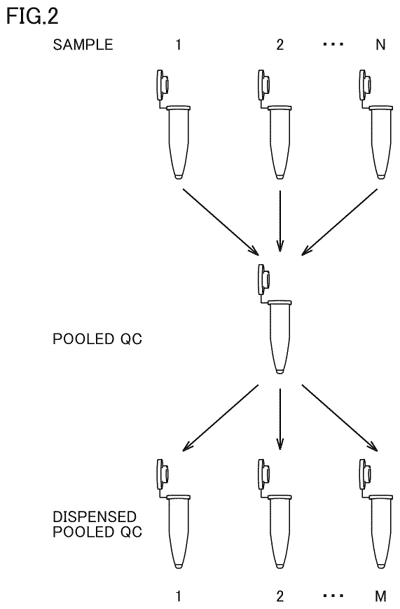

As a method of correcting the measurement error, the technique for correcting measurement values of samples based on measurement values of a standard sample called “pooled QC” as described in “Procedures for large-scale metabolic profiling of serum and plasma using gas chromatography and liquid chromatography coupled to mass spectrometry” by Dunn, Warwick B., et al., Nature protocols 6.7 (2011): 1060-1083 has been known. FIG. 2 is a diagram for illustrating a procedure of preparing a pooled QC. A case in which there are N samples to be measured is assumed. As shown in FIG. 2, a pooled QC is prepared by mixing all of the samples in equal amounts. Since all of the samples are mixed in equal amounts, an amount of each component included in the pooled QC is an average of all of the samples about this component. That is, the measurement values of the pooled QC are close to an average value of the measurement values of all of the samples, which makes it possible to prevent the measurement values of the pooled QC from significantly deviating from the measurement values of the samples. In addition, since the pooled QC is prepared by mixing all of the samples, a component included in at least any one of the samples is included in the pooled QC. Therefore, even when a component to be subjected to correction is unknown at the start of measurement, measurement values of the component can be subjected to correction processing after measurement. The prepared pooled QC is dispensed into the appropriate number of pieces. In FIG. 2, the prepared pooled QC is divided into M pieces.

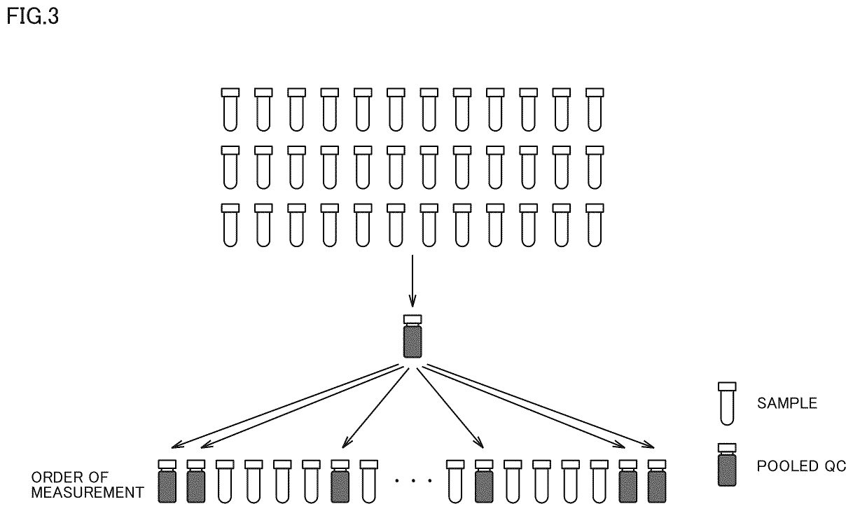

The mass spectrometer measures the pooled QC before and after measurement of the samples, and sequentially obtains measurement values of the samples and measurement values of the pooled QC. FIG. 3 is a diagram for illustrating an order of measurement of the pooled QC and the samples. In FIG. 3, a white container represents the sample to be measured, and a gray container represents the pooled QC. An operator extracts an equal amount of solution from each of thirty-six samples and prepares one pooled QC. The prepared pooled QC is divided into the appropriate number of pieces and is subjected to measurement at an appropriate timing before and after measurement of the samples. For example, as shown in FIG. 3, the mass spectrometer performs two consecutive measurements of the pooled QC before the start of measurement of the samples, and then, performs one measurement of the pooled QC every time the mass spectrometer measures four samples. After the last sample is measured, the mass spectrometer performs two consecutive measurements of the pooled QC.

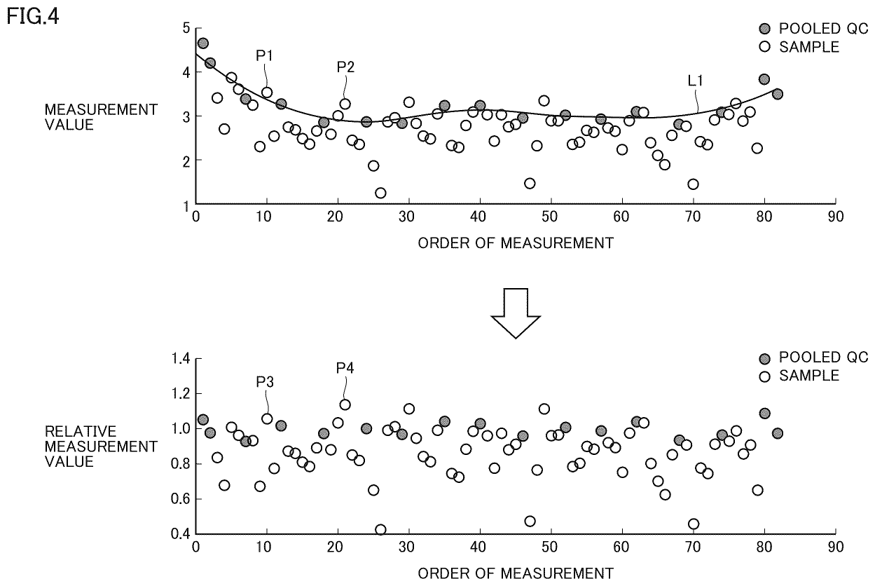

By performing a series of measurement as described above, the measurement values of the samples and the measurement values of the pooled QC are sequentially obtained. Processing device 10 corrects the measurement values of the samples based on the measurement values of the pooled QC. FIG. 4 is a diagram for illustrating a method of correcting the measurement values of the samples based on the measurement values of the pooled QC. In the graph shown in the upper part of FIG. 4, the obtained measurement values are shown in the order of measurement of the samples and the pooled QC. A white circle represents the measurement value of the sample, and a gray circle represents the measurement value of the pooled QC. Here, the 10-th measurement value of the sample indicated by a point P1 is larger than the 21-th measurement value of the sample indicated by a point P2, for example.

Ideally, the measurement values of the pooled QC should be equal to each other even if the pooled QC is measured at any timing. Therefore, in order to correct the measurement values of the samples, LOESS smoothing is performed using only the measurement values of the pooled QC, to calculate an approximate curve L1. Based on calculated approximate curve L1, the measurement values of the samples are corrected. The graph shown in the lower part of FIG. 4 shows relative measurement values of the samples after correction. The relative measurement values in this graph are normalized values when a value indicated by approximate curve L1 is 1. In the graph shown in the lower part of FIG. 4, the measurement values of the pooled QC are corrected to be substantially constant.

A point P3 represents the 10-th measurement value of the sample after correction, and a point P4 represents the 21-th measurement value of the sample after correction. Although point P1 is larger than point P2 before correction, point P3 is smaller than point P4 after correction. As described above, the use of the QC-RLSC method makes it possible to correct the measurement error and improve the accuracy of measurement.

However, the correction method using the pooled QC has some problems. (1) When the samples are solids, for example, preparation of the pooled QC may be difficult. Even if the pooled QC can be prepared, (2) all of the samples are needed to prepare the pooled QC, and thus, measurement cannot be started before the last sample is obtained. The delay in the start of measurement may cause a change in quality of the obtained samples. (3) Preparation of the pooled QC requires a task of dispensing all of the samples in equal amounts, which may lead to the longer work time of the user. (4) When an amount of each sample is small, an insufficient amount of the samples may be subjected to analysis because the samples are used to prepare the pooled QC. (5) Since a component included in a part of the samples is low in content in the pooled QC, measurement values of this component cannot in some cases be obtained in measurement of the pooled QC. (6) It is necessary to measure the pooled QC in between measurements of the samples, and thus, the number of samples that can be analyzed per unit time is small.

### Data Processing Method According to Embodiment

Thus, in a data processing method according to the present embodiment, based on measurement values of samples, processing device 10 estimates values that might be obtained when preparing a pooled QC and measuring the pooled QC at the time of measurement of the samples. Processing device 10 corrects the measurement values of the samples by regarding the estimated values as measurement values of the pooled QC. The use of the data processing method according to the present embodiment allows processing device 10 to correct the measurement values of the samples using a correction method equivalent to the QC-RLSC method, without using the actual measurement values of the pooled QC.

In the data processing method according to the present embodiment, it is unnecessary to actually prepare the pooled QC. Therefore, even when the samples are solids, the measurement values can be corrected. In addition, since it is unnecessary to prepare the pooled QC before the start of measurement of the samples, measurement can be started before all of the samples are obtained. Furthermore, the work time of the user required to prepare the pooled QC can be reduced.

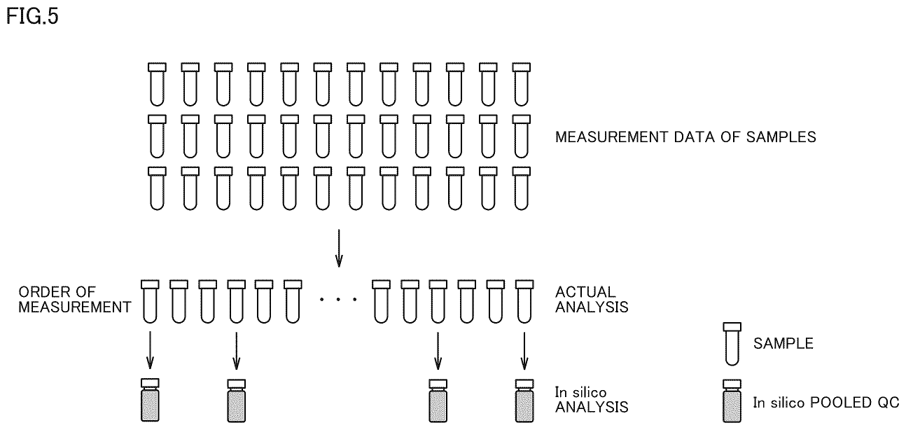

FIG. 5 is a diagram for illustrating a method of correcting the measurement values of the samples obtained by mass spectrometer 40 using the data processing method according to the present embodiment.

Referring to FIG. 5, first, mass spectrometer 40 analyzes the samples. Processing device 10 receives measurement values of a prescribed component from mass spectrometer 40. Processing device 10 may invoke the measurement values of the samples stored in memory 12, or may receive the measurement values obtained by the device other than mass spectrometer 40. Since mass spectrometer 40 sequentially measures the samples, the measurement values can be arranged in accordance with the order of measurement as shown in “actual analysis” in FIG. 5.

Next, measurement values of the pooled QC are estimated based on “measurement data of samples”. The estimation is referred to as “In silico analysis” because actual analysis processing is not performed and the estimation is performed on a computer. A value estimated by “In silico analysis” is referred to as a value of “In silico pooled QC”. The value of the In silico pooled QC is also referred to as a reference value. The reference value is a value that might be obtained when a pooled QC, which is a standard sample obtained by mixing measurement values of samples, is measured at the timing of measurement of the samples.

In the correction processing described in the comparative example, mass spectrometer 40 needs to actually measure the pooled QC. Therefore, it is physically impossible to simultaneously measure the pooled QC and the samples. In contrast, in the In silico analysis, actual measurement of the pooled QC by mass spectrometer 40 is not performed, and thus, the reference values can be calculated, assuming that the pooled QC is measured simultaneously with measurement of the samples. In FIG. 5, it is assumed that the In silico pooled QC is measured at the timing of a first measurement of the sample and at the timing of a fourth measurement of the sample. In this case, a reference value corresponding to the first measurement and a reference value corresponding to the fourth measurement are calculated. Although the reference value corresponding to the fourth measurement is a second reference value, the reference value corresponding to the fourth measurement is referred to as a fourth reference value for the sake of convenience. The reference values may be calculated for all of the measurement values, or may be selectively calculated for some of the measurement values.

Specifically, a k-th (k is a natural number) reference value I(k) is calculated in accordance with Equation (1) below.

$$\begin{matrix}[ Math . 1 ] & \\ I ( k ) = \frac{{\sum }_{i = r}^{k} exp ( - {( i - n )}^{2} ) \times P ( i )}{{\sum }_{i = r}^{k} exp ( - {( i - n )}^{2} )} & ( 1 )\end{matrix}$$

In Equation (1), P(i) represents an i-th measurement value. In Equation (1), r-th (r is a natural number satisfying r<k) to k-th measurement values are used to calculate the k-th reference value. This is because the reference value to be calculated is affected by measurement before this reference value.

In addition, in Equation (1), the measurement value is multiplied by a coefficient corresponding to the order of measurement. This is because the reference value is more greatly affected by measurement close to measurement to be subjected to calculation. Therefore, the coefficient becomes larger as the measurement value becomes closer to the k-th measurement value. A weighting function used to calculate the reference value is not limited to an exp function and may be an inverse square function, a logistic function and a hinge function.

Furthermore, k-th reference value I(k) may be calculated in accordance with Equation (2) below.

$$\begin{matrix}[ Math . 2 ] & \\ I ( k ) = {P}_{mean} + \frac{{\sum }_{i = r}^{k} exp ( - {( i - n )}^{2} ) \times ( P ( i ) - {P}_{mean} )}{{\sum }_{i = r}^{k} exp ( - {( i - n )}^{2} )} & ( 2 )\end{matrix}$$

In Equation (2), Pmean represents an average value of first to Q-th measurement values.

In Equations (1) and (2), r is preferably 1. When r is 1, the number of the measurement values used to calculate the k-th reference value is maximized and the accuracy of calculation of the reference value can be improved.

Processing device 10 performs LOESS smoothing on the reference values calculated as described above, to derive an approximate curve. Processing device 10 normalizes the measurement values of the samples such that the derived approximate curve has a value of 1 at the time of measurement of each sample. Since the approximate curve is calculated, at least two or more reference values need to be calculated.

In measurement by a mass spectrometer, an amount of each of a plurality of components included in samples can be determined in one measurement. In such a case, measurement values are corrected for each component.

In addition, in a chromatogram created by a mass spectrometer, the retention time may stochastically vary depending on measurement, even in the case of the same component. In such a case, the accuracy of obtained measurement values varies depending on measurement. Thus, in the above-described correction, a confidence interval may be calculated and corrected measurement values may be obtained with the confidence interval.

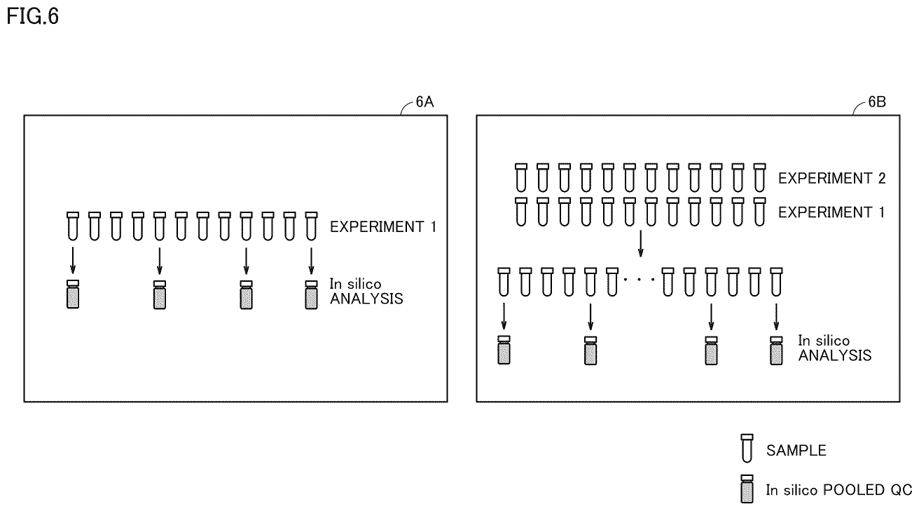

In the method described in the present embodiment, all of the samples do not necessarily need to be collected before the start of measurement. Therefore, according to the data processing method, so-called inter-batch correction for correcting measurement values of samples included in different batches can be performed. FIG. 6 is a diagram for illustrating a data processing method when experimental data is added. As shown in a box 6A of FIG. 6, a case in which a user conducts an experiment 1 and obtains twelve samples is assumed. The user measures the samples to obtain measurement values and corrects the obtained measurement values by In silico analysis. In this case, the measurement of the twelve samples is referred to a batch. Thereafter, the user conducts an experiment 2 and obtains new twelve samples different from the samples obtained in experiment 1. A measurement of the twelve samples obtained in experiment 2 is a batch different from the batch in experiment 1. When the correction method described in the comparative example is used to correct measurement values of the samples obtained in experiment 2, the user needs to prepare a pooled QC by mixing the samples obtained in experiment 1 and the samples obtained in experiment 2, and then, again measure the samples obtained in experiment 1. In contrast, when the method described in the present embodiment is used to correct the measurement values of the samples obtained in experiment 2, the measurement values of the samples obtained in experiment 2 and the measurement values of the samples already obtained in experiment 1 can be subjected to correction processing together, as shown in a box 6B. Therefore, according to the method described in the present embodiment, even when measurement values of samples included in a different batch are corrected, the measurement values can be corrected without again measuring samples that have already been measured.

### Processing of Measurement Values

Processing of the measurement values obtained by mass spectrometer 40 may be performed before the measurement values are corrected as described above. Examples of the processing include a process of detecting and excluding an abnormal value, and a process of imputing a missing value.

The process of detecting and excluding an abnormal value is a process of detecting and excluding a measurement value that significantly deviates from the other measurement values due to an error during measurement, of the measurement values. Cluster analysis using a score plot from PCA of all samples, and a detection method using a box plot can, for example, be used to detect the abnormal value.

The process of imputing a missing value is a process of imputing a missing value using a statistical method. The missing value refers to a measurement value corresponding to a sample excluded as an abnormal value in measurement data, and a measurement value corresponding to a sample whose measurement value is not recorded in measurement data. Examples of the statistical method of imputing a missing value include a single imputation method and a multiple imputation method.

The above-described processing of the measurement values makes it possible to prevent the abnormal value and the missing value from affecting calculation of the reference values. As a result, it is possible to prevent the reference values from taking abnormal values and affecting the approximate curve, and thus, the accuracy of correction of the measurement values can be improved.

### Flow of Data Processing

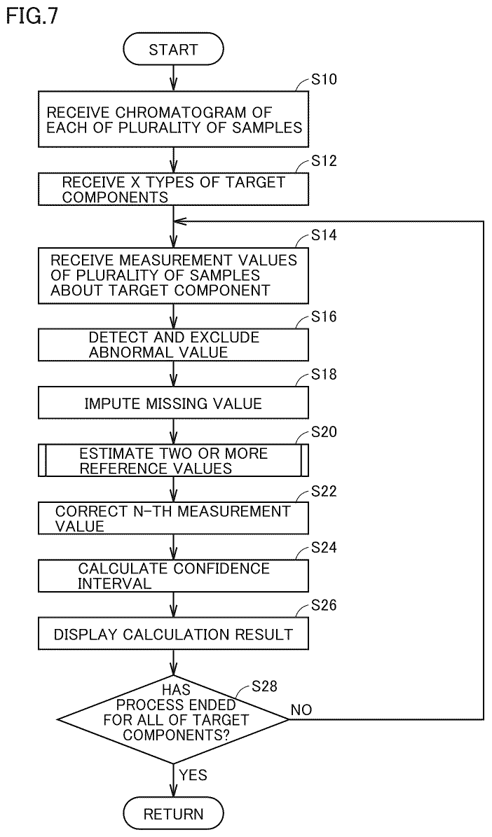

FIG. 7 shows a flowchart of an example of data processing of measurement data obtained by measuring the samples with mass spectrometer 40. In an implementation, a data processing subroutine in FIG. 7 is called from a main routine and executed when the processor of processing device 10 executes correction program 121. Processing device 10 is considered as an example of an information processing device.

In FIG. 7, a case of correcting an N-th (N is an integer equal to or greater than 3) measurement value, of measurement values of a prescribed component obtained by measuring a plurality of samples, is assumed.

In step S10, processing device 10 receives a chromatogram of each of the plurality of samples obtained by mass spectrometer 40.

In step S12, processing device 10 receives, from a user, X types of target components to be subjected to correction.

In step S14, processing device 10 extracts a plurality of measurement values of one of the X types of target components from the chromatogram received in step S10, and receives the extracted plurality of measurement values.

In step S16, processing device 10 detects an abnormal value from the plurality of measurement values received in step S14, and excludes the detected abnormal value.

In step S18, processing device 10 imputes a missing value included in the plurality of measurement values received in step S14.

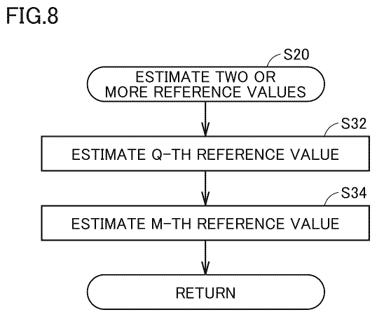

In step S20, processing device 10 estimates reference values, the reference values being values obtained when a standard sample obtained by mixing the plurality of samples is measured at the time of two or more measurements. FIG. 8 shows a reference value estimation process subroutine in step S20.

Referring to FIG. 8, in step S32, processing device 10 estimates a Q-th (Q is an integer satisfying N<Q) reference value. The Q-th reference value is calculated in accordance with Equation (1) or Equation (2), for example.

In step S34, processing device 10 estimates an M-th (M is a natural number satisfying M<Q) reference value. The M-th reference value is calculated in accordance with Equation (1) or Equation (2), for example. In step S20, processing device 10 estimates two or more reference values including the Q-th reference value and the M-th reference value. Thereafter, processing device 10 ends the reference value estimation process subroutine and returns the control to FIG. 7.

Referring to FIG. 7, in step S22, processing device 10 calculates an approximate curve using the two or more reference values including the Q-th reference value and the M-th reference value, and corrects the N-th measurement value.

In step S24, processing device 10 calculates a confidence interval of the measurement value corrected in step S22.

In step S26, processing device 10 causes display device 30 to display the corrected measurement value calculated in step S22 and the confidence interval calculated in step S24.

In step S28, processing device 10 determines whether the process has ended for the X types of target components received in step S12. When the process has ended for all of the target components (YES in step S28), processing device 10 ends the data processing subroutine and returns the process to the main routine. Otherwise (NO in step S28), processing device 10 returns the process to step S14.

In the above-described data processing, processing device 10 receives the target components from the user in step S12. However, instead of receiving the target components from the user, processing device 10 may perform data processing on all of the peaks present in the chromatograms received in step S10.

The two or more reference values used to calculate the approximate curve need to include at least a reference value before the measurement value to be corrected and a reference value after the measurement value to be corrected. As described with reference to FIG. 8, the reference values used to calculate the approximate curve need to include the M-th reference value and the Q-th reference value. The approximate curve may be calculated using only these two reference values, or may be calculated using the reference values corresponding to all of the measurements.

In the above-described data processing, the user does not need to prepare a pooled QC. Therefore, the data processing is applicable to analysis of a sample for which preparation of a pooled QC is difficult. Examples of the sample for which preparation of the pooled QC is difficult include a solid sample. Examples of the solid sample include silicon rubber.

In addition, by using the data processing, analysis of the samples can be started before all of the samples are collected. Therefore, the time from collection of the samples to the completion of analysis can be reduced. In addition, the data processing is also applicable to measurement of a substance that is highly likely to be modified and aggregated due to repeated freezing and unfreezing, for example. In the case of preparing a pooled QC, a first collected sample needs to be stored without being subjected to measurement, until the last sample is collected. Generally, a biologically originated sample is stored in a frozen manner. However, the sample may include a substance that is highly likely to be modified and aggregated due to repeated freezing and unfreezing, and thus, avoiding freezing and unfreezing of the sample is preferable.

In the data processing, measurement of the samples can be started before collection of all of the samples is completed, and thus, the number of times of freezing and unfreezing of the samples can be reduced. Therefore, the data processing is also applicable to analysis of a substance that is highly likely to be modified and aggregated due to repeated freezing and unfreezing. Examples of the substance that is highly likely to be modified and aggregated due to repeated freezing and unfreezing include insulin and mucin.

In the above-described data processing, preparation of a pooled QC is unnecessary, and thus, the work time of the user required to prepare a pooled QC can be reduced. In addition, in the above-described data processing, it is unnecessary to use a part of the obtained samples for preparation of a pooled QC, and thus, a whole amount of the obtained samples can be subjected to measurement.

In addition, in the above-described data processing, measurement values of a component included only in a part of the samples can also be corrected based on the obtained measurement values.

Furthermore, in the above-described data processing, measurement of a pooled QC is unnecessary, and thus, the number of samples that can be analyzed per unit time can be increased.

In the data processing method according to the present disclosure, a computer can perform calculation related to correction of measurement values of samples using a value of an In silico pooled QC calculated using the measurement values of the samples to be measured, without obtaining measurement values of a pooled QC that has been used in calculation for correcting a measurement error of measurement values obtained by the analysis device. Thus, even when the computer cannot obtain the measurement values of the pooled QC, the computer can perform calculation related to correction processing of the measurement values of the samples.

### ASPECTS

It will be appreciated by a person skilled in the art that the illustrative embodiments described above provide specific examples of the following aspects.

(Clause 1) A data processing method according to an aspect is a data processing method performed by a computer to correct an N-th (N is an integer satisfying 3≤N) measurement value, of a plurality of measurement values of a prescribed component obtained by measuring a plurality of samples with an analysis device, the computer including a processor and an interface, the data processing method including: receiving, by the interface, the plurality of measurement values obtained by the analysis device; estimating, by the processor, reference values, the reference values being values obtained when a standard sample obtained by mixing the plurality of samples is measured at the time of two or more measurements including at least M-th and Q-th (M and Q are natural numbers satisfying M<N<Q) measurements; and correcting, by the processor, the N-th measurement value using the reference values, wherein the estimating reference values may include: estimating, by the processor, a Q-th reference value using T-th (T is a natural number satisfying T<Q) to Q-th measurement values, the Q-th reference value being a value obtained when the standard sample is measured at the time of the Q-th measurement; and estimating, by the processor, an M-th reference value using L-th (L is a natural number satisfying L<M) to M-th measurement values, the M-th reference value being a value obtained when the standard sample is measured at the time of the M-th measurement.

In the data processing method according to clause 1, the measurement values obtained by the analysis device can be corrected without using a pooled QC.

(Clause 2) In the data processing method according to clause 1, the estimating a Q-th reference value may include multiplying a measurement value obtained at the time of a measurement closer in measurement order to the Q-th measurement by a larger coefficient, thereby assigning weight to the measurement value, and the estimating an M-th reference value may include multiplying a measurement value obtained at the time of a measurement closer in measurement order to the M-th measurement by a larger coefficient, thereby assigning weight to the measurement value.

In the data processing method according to clause 2, the measurement values obtained by the analysis device can be corrected without using a pooled QC. Each of the reference values used for correction is estimated by multiplying a measurement value obtained at the time of a measurement closer in measurement order to a measurement of the reference value by the larger coefficient, and assigning weight to the measurement value.

(Clause 3) In the data processing method according to clause 1, the estimating reference values may further include calculating an average value, the average value being an average of the plurality of measurement values, and the average value may be used in the estimating a Q-th reference value and the estimating an M-th reference value.

In the data processing method according to clause 3, the measurement values obtained by the analysis device can be corrected without using a pooled QC. The average value of the plurality of measurement values is used as each of the reference values.

(Clause 4) In the data processing method according to clause 1 or 2, the estimating a Q-th reference value may include calculating the Q-th reference value in accordance with Equation (3) by substituting T into r and Q into k, when P(k) represents a k-th measurement value and I(k) represents a k-th reference value,

$$\begin{matrix}[ Math . 3 ] & \\ I ( k ) = \frac{{\sum }_{i = r}^{k} exp ( - {( i - n )}^{2} ) \times P ( i )}{{\sum }_{i = r}^{k} exp ( - {( i - n )}^{2} )} , & ( 3 )\end{matrix}$$

and the estimating an M-th reference value may include calculating the M-th reference value in accordance with Equation (3) by substituting L into r and M into k.

In the data processing method according to clause 4, the measurement values obtained by the analysis device can be corrected without using a pooled QC. The reference values used for correction are calculated in accordance with Equation (3).

(Clause 5) In the data processing method according to clause 1 or 3, the estimating a Q-th reference value may include calculating the Q-th reference value in accordance with Equation (4) by substituting T into r and Q into k, when P(k) represents a k-th measurement value, I(k) represents a k-th reference value, and Pmean represents an average value of the plurality of measurement values,

$$\begin{matrix}[ Math . 4 ] & \\ I ( k ) = {P}_{mean} + \frac{{\sum }_{i = r}^{k} exp ( - {( i - n )}^{2} ) \times ( P ( i ) - {P}_{mean} )}{{\sum }_{i = r}^{k} exp ( - {( i - n )}^{2} )} , & ( 4 )\end{matrix}$$

and
the estimating an M-th reference value may include calculating the M-th reference value in accordance with Equation (4) by substituting L into r and M into k.

In the data processing method according to clause 5, the measurement values obtained by the analysis device can be corrected without using a pooled QC. The reference values used for correction are calculated in accordance with Equation (4).

(Clause 6) In the data processing method according to any one of clauses 1 to 5, the T and the L may be 1.

In the data processing method according to clause 6, a first measurement value to a measurement value having a prescribed order are used to calculate a reference value having the prescribed order. As a result, the accuracy of calculation of the reference value can be improved.

(Clause 7) In the data processing method according to any one of clauses 1 to 6, the correcting the N-th measurement value may include correcting the N-th measurement value based on an approximate curve calculated by performing LOESS smoothing using the Q-th reference value and the M-th reference value.

In the data processing method according to clause 7, the measurement value is corrected based on the approximate curve calculated by performing LOESS smoothing.

(Clause 8) The data processing method according to any one of clauses 1 to 7 may further include detecting and excluding an abnormal value of the measurement values.

In the data processing method according to clause 8, the abnormal value included in the measurement values is excluded, and thus, the accuracy of correction of the measurement values can be improved.

(Clause 9) In the data processing method according to clause 8, the abnormal value may be detected by using one of a cluster analysis method using a score plot from PCA of the measurement values, and a detection method using a box plot.

In the data processing method according to clause 9, the abnormal value included in the measurement values is detected and excluded by using one of the cluster analysis method using the score plot from PCA of the measurement values, and the detection method using the box plot, and thus, the accuracy of correction of the measurement values can be improved.

(Clause 10) The data processing method according to any one of clauses 1 to 9 may further include imputing a missing value of the measurement values.

In the data processing method according to clause 10, the missing value included in the measurement values is imputed, and thus, the accuracy of correction of the measurement values can be improved.

(Clause 11) In the data processing method according to clause 10, the imputing a missing value may include imputing the missing value using one of a single imputation method and a multiple imputation method.

In the data processing method according to clause 11, the missing value included in the measurement values is imputed using one of the single imputation method and the multiple imputation method, and thus, the accuracy of correction of the measurement values can be improved.

(Clause 12) The data processing method according to any one of clauses 1 to 11 may further include deriving a confidence interval of the N-th measurement value corrected in the correcting the N-th measurement value.

In the data processing method according to clause 12, the user can recognize the confidence interval of the corrected value and can easily determine whether to use the data in analysis.

(Clause 13) In the data processing method according to any one of clauses 1 to 12, the analysis device may be a mass spectrometer.

In the data processing method according to clause 13, the measurement values obtained by the mass spectrometer can be corrected without using a pooled QC.

(Clause 14) An information processing device according to an aspect is an information processing device that corrects a measurement value obtained by measurement with an analysis device. The information processing device includes: at least one or more processors; and a memory configured to access the one or more processors, wherein the memory is configured to store one or more instructions executed by the one or more processors, and the processor is configured to, by executing the one or more instructions, receive measurement values of a prescribed component obtained by measuring a plurality of samples with the analysis device, estimate reference values, the reference values being values obtained when a standard sample obtained by mixing the plurality of samples is measured at the time of two or more measurements including at least M-th and Q-th (M and Q are natural numbers satisfying M<Q) measurements, and correct an N-th (N is a natural number satisfying M<N<Q) measurement value using the reference values, and the processor may be configured to, when estimating the reference values, estimate a Q-th reference value using T-th (T is a natural number satisfying T<Q) to Q-th measurement values, the Q-th reference value being a value obtained when the standard sample is measured at the time of the Q-th measurement, and estimate an M-th reference value using L-th (L is a natural number satisfying L<M) to M-th measurement values, the M-th reference value being a value obtained when the standard sample is measured at the time of the M-th measurement.

In the information processing device according to clause 14, the measurement values obtained by the analysis device can be corrected without using a pooled QC.

(Clause 15) A program according to an aspect, by being executed by a processor mounted on a computer, causes the computer to: receive measurement values of a prescribed component obtained by measuring a plurality of samples with an analysis device; estimate reference values, the reference values being values obtained when a standard sample obtained by mixing the plurality of samples is measured at the time of two or more measurements including at least M-th and Q-th (M and Q are natural numbers satisfying M<Q) measurements; and correct an N-th (N is a natural number satisfying M<N<Q) measurement value using the reference values, and the program may cause the computer to, when estimating the reference values, estimate a Q-th reference value using T-th (T is a natural number satisfying T<Q) to Q-th measurement values, the Q-th reference value being a value obtained when the standard sample is measured at the time of the Q-th measurement, and estimate an M-th reference value using L-th (L is a natural number satisfying L<M) to M-th measurement values, the M-th reference value being a value obtained when the standard sample is measured at the time of the M-th measurement.

In the program according to clause 15, the measurement values obtained by the analysis device can be corrected without using a pooled QC.

Although the embodiment of the present disclosure has been described, it should be understood that the embodiment disclosed herein is illustrative and non-restrictive in every respect. The scope of the present disclosure is defined by the terms of the claims, and is intended to include any modifications within the scope and meaning equivalent to the terms of the claims.

What is claimed is:

1. A data processing method performed by a computer to correct an N-th (N is an integer satisfying 3≤N) measurement value, of a plurality of measurement values of a prescribed component obtained by measuring a plurality of samples with an analysis device,
the computer including a processor and an interface,
the data processing method comprising:
receiving, by the interface, the plurality of measurement values obtained by the analysis device;
estimating, by the processor, reference values, the reference values being values obtained when a standard sample obtained by mixing the plurality of samples is measured at the time of two or more measurements including at least M-th and Q-th (M and Q are natural numbers satisfying M<N<Q) measurements; and
correcting, by the processor, the N-th measurement value using the reference values, wherein
the estimating reference values includes:
estimating, by the processor, a Q-th reference value using T-th (T is a natural number satisfying T<Q) to Q-th measurement values, the Q-th reference value being a value obtained when the standard sample is measured at the time of the Q-th measurement; and
estimating, by the processor, an M-th reference value using L-th (L is a natural number satisfying L<M) to M-th measurement values, the M-th reference value being a value obtained when the standard sample is measured at the time of the M-th measurement.

2. The data processing method according to claim 1, wherein
the estimating a Q-th reference value includes multiplying a measurement value obtained at the time of a measurement closer in measurement order to the Q-th measurement by a larger coefficient, thereby assigning weight to the measurement value, and
the estimating an M-th reference value includes multiplying a measurement value obtained at the time of a measurement closer in measurement order to the M-th measurement by a larger coefficient, thereby assigning weight to the measurement value.

3. The data processing method according to claim 1, wherein
the estimating reference values further includes calculating an average value, the average value being an average of the plurality of measurement values, and
the average value is used in the estimating a Q-th reference value and the estimating an M-th reference value.

4. The data processing method according to claim 1, wherein
the estimating a Q-th reference value includes calculating the Q-th reference value in accordance with Equation (1) by substituting T into r and Q into k, when P(k) represents a k-th measurement value and I(k) represents a k-th reference value,
$$\begin{matrix}[ Math . 1 ] & \\ I ( k ) = \frac{{\sum }_{i = r}^{k} exp ( - {( i - n )}^{2} ) \times P ( i )}{{\sum }_{i = r}^{k} exp ( - {( i - n )}^{2} )} , & ( 1 )\end{matrix}$$
and
the estimating an M-th reference value includes calculating the M-th reference value in accordance with Equation (1) by substituting L into r and M into k.

5. The data processing method according to claim 1, wherein
the estimating a Q-th reference value includes calculating the Q-th reference value in accordance with Equation (2) by substituting T into r and Q into k, when P(k) represents a k-th measurement value, I(k) represents a k-th reference value, and Pmean represents an average value of the plurality of measurement values,
[Math. 2]
$$I ( k ) = {P}_{mean} + \frac{{\sum }_{i = r}^{k} exp ( - {( i - n )}^{2} ) \times ( P ( i ) - {P}_{mean} )}{{\sum }_{i = r}^{k} exp ( - {( i - n )}^{2} )} , \qquad ( 2 )$$
and
the estimating an M-th reference value includes calculating the M-th reference value in accordance with Equation (2) by substituting L into r and M into k.

6. The data processing method according to claim 1, wherein
the T and the L are 1.

7. The data processing method according to claim 1, wherein
the correcting the N-th measurement value includes correcting the N-th measurement value based on an approximate curve calculated by performing LOESS smoothing using the Q-th reference value and the M-th reference value.

8. The data processing method according to claim 1, further comprising
detecting and excluding an abnormal value of the measurement values.

9. The data processing method according to claim 8, wherein
the abnormal value is detected by using one of a cluster analysis method using a score plot from PCA of the measurement values, and a detection method using a box plot.

10. The data processing method according to claim 1, further comprising
imputing a missing value of the measurement values.

11. The data processing method according to claim 10, wherein
the imputing a missing value includes imputing the missing value using one of a single imputation method and a multiple imputation method.

12. The data processing method according to claim 1, further comprising
deriving a confidence interval of the N-th measurement value corrected in the correcting the N-th measurement value.

13. The data processing method according to claim 1, wherein
the analysis device is a mass spectrometer.

14. An information processing device that corrects a measurement value obtained by measurement with an analysis device, the information processing device comprising:
at least one or more processors; and
a memory configured to access the one or more processors, wherein
the memory is configured to store one or more instructions executed by the one or more processors,
the processor is configured to, by executing the one or more instructions,
receive measurement values of a prescribed component obtained by measuring a plurality of samples with the analysis device,
estimate reference values, the reference values being values obtained when a standard sample obtained by mixing the plurality of samples is measured at the time of two or more measurements including at least M-th and Q-th (M and Q are natural numbers satisfying M<Q) measurements, and
correct an N-th (N is a natural number satisfying M<N<Q) measurement value using the reference values, and
the processor is configured to, when estimating the reference values,
estimate a Q-th reference value using T-th (T is a natural number satisfying T<Q) to Q-th measurement values, the Q-th reference value being a value obtained when the standard sample is measured at the time of the Q-th measurement, and
estimate an M-th reference value using L-th (L is a natural number satisfying L<M) to M-th measurement values, the M-th reference value being a value obtained when the standard sample is measured at the time of the M-th measurement.

15. A non-transitory computer readable medium having a program recorded thereon, wherein
the program, by being executed by a processor of a computer, causes the computer to:
receive measurement values of a prescribed component obtained by measuring a plurality of samples with an analysis device;
estimate reference values, the reference values being values obtained when a standard sample obtained by mixing the plurality of samples is measured at the time of two or more measurements including at least M-th and Q-th (M and Q are natural numbers satisfying M<Q) measurements; and
correct an N-th (N is a natural number satisfying M<N<Q) measurement value using the reference values, and
the program causes the computer to, when estimating the reference values,
estimate a Q-th reference value using T-th (T is a natural number satisfying T<Q) to Q-th measurement values, the Q-th reference value being a value obtained when the standard sample is measured at the time of the Q-th measurement, and
estimate an M-th reference value using L-th (L is a natural number satisfying L<M) to M-th measurement values, the M-th reference value being a value obtained when the standard sample is measured at the time of the M-th measurement.
